(19) United States
(12) Patent Application Publication
(10) Pub. No.: US 2016/0100826 A1
(43) Pub. Date: Apr. 14, 2016

(54) PREDICTED MENSTRUATION START DATE CALCULATION APPARATUS, PROGRAM, AND BODY ANALYSIS APPARATUS

(71) Applicants: OMRON HEALTHCARE CO., LTD., Kyoto (JP); OMRON CORPORATION, Kyoto (JP)

(72) Inventors: Mayumi Akatsuka, Kyoto-shi, Kyoto (JP); Naoki Tsuchiya, Kyoto-shi, Kyoto (JP); Hiroshi Nakajima, Kyoto-shi, Kyoto (JP); Akiyoshi Fujisaki, Kyoto (JP); Yutaka Otsubo, Kyoto (JP)

(73) Assignees: OMRON HEALTHCARE CO., LTD., Kyoto (JP); OMRON CORPORATION, Kyoto-shi (JP)

(21) Appl. No.: 14/921,876

(22) PCT Filed: Apr. 17, 2014
(86) PCT No.: PCT/JP2014/060922

§ 371 Date: Oct. 23, 2015

(30) Foreign Application Priority Data
Apr. 25, 2013 (JP) 2013-092818

(51) Int. Cl. A61B 10/00; A61B 5/00; A61B 5/01

(52) U.S. Cl. CPC A61B 10/0012; A61B 5/01; A61B 5/4318; A61B 5/742; A61B 2010/0019

(57) ABSTRACT

A predicted menstruation start date calculation apparatus includes a date information acquisition unit that acquires a number of menstrual cycle days and a number of high temperature phase days over multiple menstrual cycles of a woman, a type classification unit that performs classification into a specific type indicating that the number of menstrual cycle days is unstable and the number of high temperature phase days is stable, and into one or more types other than the specific type, and a calculation unit that calculates the predicted menstruation start date based on the classification by the type classification unit.

### TECHNICAL FIELD

Embodiments of the claimed invention relate to a predicted menstruation start date calculation apparatus, and more specifically relates to an apparatus that calculates a woman's predicted menstruation start date.

Also, embodiments of the claimed invention relate to a program for causing a computer to execute a method for calculating a woman's predicted menstruation start date.

Also, embodiments of the claimed the invention relate to a body analysis apparatus that analyzes a body.

### BACKGROUND ART

Conventionally, a basal thermometer disclosed in Patent Document 1 (JP 4240632B) for example has been known as an apparatus that calculates a woman's predicted menstruation start date. With this basal thermometer, a menstruation start date that is n cycles in the future is calculated using the following equation:

(Most recent menstruation start date+n×average number of effective menstrual cycle days)

Also, with a basal thermometer disclosed in Patent Document 2 (JP 3705470B), if a woman is in a low temperature phase on the day of performing measurement processing, the predicted menstruation start date is calculated using the following equation (here, the possible period is the equivalent of 1.5σ divided by 2, assuming that the distribution of menstrual cycles used to calculate the average menstrual cycle is a normal distribution):

Next menstruation date=between (most recent menstruation start date+average menstrual cycle−possible period), and (most recent menstruation start date+average menstrual cycle+possible period)

On the other hand, if the woman is in a high temperature phase on the day of performing calculation processing, the predicted menstruation start date is calculated using the following equation (here, the possible period is the equivalent of 1.5σ divided by 2, assuming that the distribution of high temperature phase periods used to calculate the average high temperature phase period is a normal distribution):

Next menstruation date=between (most recent ovulation date+average high temperature phase period−possible period), and (most recent ovulation date+average high temperature phase period+possible period).

### CITATION LIST

### Patent Literature

Patent Document 1: JP 4240632B

Patent Document 2: JP 3705470B

### SUMMARY OF INVENTION

However, with both of the above-described basal thermometers, the predicted menstruation start date is calculated uniformly regardless of whether or not the number of menstrual cycle days of the woman who is the measurement subject is stable, and regardless of whether or not the number of high temperature phase days is stable.

For example, with the basal thermometer disclosed in Patent Document 1, even if the number of menstrual cycle days of the woman who is the measurement subject is unstable, a menstruation start date that is n cycles in the future is calculated uniformly using the following equation:

(Most recent menstruation start date+n×average number of effective menstruation cycle days)

Also, with the basal thermometer disclosed in Patent Document 2, even if the number of high temperature phase days (high temperature phase period) of the woman who is the measurement subject is unstable, as long as she is in the high temperature phase on the day when the calculation processing is performed, the predicted menstruation start date is calculated uniformly using the following equation:

Next menstruation date−between (most recent ovulation date+average high temperature phase period−possible period), and (most recent ovulation date+average high temperature phase period+possible period)

For this reason, in both of the above-described basal thermometers, the precision of the predicted menstruation start date deteriorates depending on whether or not the number of menstrual cycle days of the woman who is the measurement subject is stable, and whether or not the number of high temperature phase days is stable.

In view of this, one or more embodiments of the claimed invention provides predicted menstruation start date calculation apparatus capable of precisely calculating a predicted menstruation start date according to types determined based on whether or not the number of menstrual cycle days of the woman who is the measurement subject is stable, and whether or not the number of high temperature phase days is stable.

Also, one or more embodiments of the claimed invention provides a program for causing a computer to execute a method for calculating a woman's predicted menstruation start date, according to which it is possible to precisely calculate the predicted menstruation start date according to types determined based on whether or not the number of menstrual cycle days of the woman who is the measurement subject is stable, and whether or not the number of high temperature phase days is stable.

Also, one or more embodiments of the claimed invention provides a body analysis apparatus capable of analyzing a body precisely, according to a type relating to transition between phases that each indicate an internal state of a body.

The predicted menstruation start date calculation apparatus according to one or more embodiments of the claimed invention includes:

a date information acquisition unit configured to acquire a number of menstrual cycle days and a number of high temperature phase days over a plurality of menstrual cycles of a woman;

a type classification unit configured to, based on whether or not the number of menstrual cycle days of the woman falls within a predetermined range and whether or not the number of high temperature phase days of the woman falls within a predetermined range, perform classification into a specific type indicating that the number of menstrual cycle days is unstable and the number of high temperature phase days is stable, and into one or more types other than the specific type; and

a calculation unit configured to, if the type into which the woman is classified by the type classification unit is the specific type, calculate a predicted menstruation start date by adding a number of days obtained based on the number of high temperature phase days to the woman's most recent ovulation date, and if the type into which the woman is classified by the type classification unit is a type other than the specific type, calculate the predicted menstruation start date based on the woman's most recent menstruation start date and the number of menstrual cycle days.

In the present specification, “woman” refers in a broad sense to a female who menstruates, and is not limited to a female age 20 or over or a married female.

“Menstrual cycle” refers to a period of time spanning from a menstruation start date to the day before the next menstruation start date (or a predicted menstruation start date).

“Number of high temperature phase days” means the number of high temperature phase days in a case where the basal body temperature curve exhibits two phases, namely a low temperature phase and a high temperature phase. In the present specification, it is assumed that the day before the high temperature phase period (the day before the first day of the high temperature phase period) is the ovulation date (in this sense, the ovulation date is an estimated date. This applies throughout the present specification).

The “number of menstrual cycle days” and “number of high temperature phase days” over a plurality of menstrual cycles refer to values obtained by averaging the “number of menstrual cycle days” and the “number of high temperature phase days” over a plurality of past menstrual cycles.

A “predetermined range” for a number of menstrual cycle days refers to a range in which the standard deviation of the number of menstrual cycle days for the most recent three to five cycles is less than or equal to a statistical survey value determined by a gynecologist, or the like.

With regard to the number of high temperature phase days, the “predetermined range” refers to a range in which the standard deviation of the number of high temperature phase days of the most recent three to five cycles is less than or equal to a statistical survey value determined by a gynecologist, or the like.

In order to “acquire” the “menstruation start date” and the “number of menstrual cycle days”, a user (typically refers to the woman, but may be medical personnel such as a doctor or a nurse, for example) may directly input the “menstruation start date” and the “number of menstrual cycle days” via the operation unit (touch panel, keyboard, mouse, etc.), for example. Also, the woman's basal body temperature data for each day, which was measured using the basal thermometer, may be acquired and the basal body temperature data may be used to estimate and obtain the menstruation start date and number of menstrual cycle days. Furthermore, the menstruation start date and number of menstrual cycle days estimated based on the basal body temperature data in this way may be received from outside of the apparatus.

In order to “acquire” the “ovulation late.” and the “number of high temperature phase days”, it is sufficient that the woman's basal body temperature data for each day, which was measured using the basal thermometer, is acquired, and the ovulation date and number of high temperature phase days are obtained by estimation based on the basal body temperature data. Alternatively, the ovulation date and number of high temperature phase days estimated based on the basal body temperature data in this way may be received from outside of the apparatus.

Note that the basal body temperature data can be acquired from the thermometer via a communication unit (near field communication, etc.), for example.

Examples of types other than the specific type (type indicating that the number of menstrual cycle days is unstable and the number of high temperature phase days is stable) include a type indicating that the number of menstrual cycle days and the number of high temperature phase days are both stable, a type indicating that the number of menstrual cycle days is stable and the number of high temperature phase days is unstable, and a type indicating that the number of menstrual cycle days and the number of high temperature phase days are both unstable.

With the predicted menstruation start date calculation apparatus of this invention, the date information acquisition unit acquires the number of menstrual cycle days and number of high temperature phase days over a plurality of menstrual cycles of the woman via an operation unit (touch panel, keyboard, mouse, etc.), for example. Based on whether or not the number of menstrual cycle days of the woman falls within a predetermined range and whether or not the number of high temperature phase days of the woman falls within a predetermined range, the type classification unit performs classification into a specific type indicating that the number of menstrual cycle days is unstable and the number of high temperature phase days is stable, and into one or more types other than the specific type. If the type into which the woman is classified by the type classification unit is the specific type, the calculation unit calculates the predicted menstruation start date by adding a number of days obtained-based on the number of high temperature phase days to the woman's most recent ovulation date. Here, if the type into which the woman is classified by the type classification unit is the specific type, the number of high temperature phase days of the woman is stable, and therefore the predicted menstruation start date can be calculated precisely. On the other hand, if the type into which the woman is classified by the type classification unit is a type other than the specific type, the calculation unit calculates the predicted menstruation start date based on the woman's most recent menstruation start date and the number of menstrual cycle days. Here, if the type into which the woman is classified by the type classification unit is, for example, a type indicating that the number of menstrual cycle days and the number of high temperature phase days are both stable, or a type indicating that the number of menstrual cycle days is stable and the number of high temperature phase days is unstable, the number of menstrual cycle days of the woman is stable, and therefore the predicted menstruation start date can be calculated precisely. Note that if the woman belongs to a type indicating that the number of menstrual cycle days and the number of high temperature phase days are both unstable, it is intrinsically difficult to precisely calculate the woman's predicted menstruation start date.

With a predicted menstruation start date calculation apparatus according to an embodiment, if the woman belongs to the specific type, the calculation unit calculates the predicted menstruation start date based on the woman's most recent menstruation start date and the number of menstrual cycle days, and thereafter calculates the predicted menstruation start date by adding a number of days obtained based on the number of high temperature phase days to the woman's most recent ovulation date, and updates the predicted menstruation start date.

With the predicted menstruation start date calculation apparatus according to this embodiment, if the woman belongs to the specific type, the calculation unit calculates the predicted menstruation start date based on the woman's most recent menstruation start date and the number of menstrual cycle days, and thereafter calculates the predicted menstruation start date by adding a number of days obtained based on the number of high temperature phase days to the woman's most recent ovulation date, and updates the predicted menstruation start date. Accordingly, regardless of whether the woman belongs to the specific type or a type other than the specific type, the user can find out the next predicted menstruation start date on the most recent menstruation start date.

Furthermore, if the woman belongs to the specific type, the calculation unit calculates the predicted menstruation start date by adding a number of days obtained based on the number of high temperature phase days to the woman's most recent ovulation date and updates the predicted menstruation start date. Accordingly, if the woman belongs to the specific type, the user can find out the next predicted menstruation start date on the day when the ovulation date is detected, with greater precision.

A predicted menstruation start date calculation apparatus according to an embodiment further includes a display unit configured to display the woman's predicted menstruation start date on a display screen as characters indicating a date or as an icon on a calendar.

With the predicted menstruation start date calculation apparatus according to this embodiment, the display unit displays the woman's predicted menstruation start date on the display screen as characters or an icon on a calendar indicating a date. Accordingly, the user can easily find out the next predicted menstruation start date by looking at the display of the display screen.

With a predicted menstruation start date calculation apparatus according to an embodiment, in an emphasized manner on the display screen, the display unit displays the woman's predicted menstruation start date that was calculated by adding the number of days obtained based on the number of high temperature phase days to the woman's most recent ovulation date, in comparison to the woman's predicted menstruation start date that was calculated based on the woman's most recent menstruation start date and the number of menstrual cycle days.

With the predicted menstruation start date calculation apparatus according to this embodiment, in an emphasized manner on the display screen, the display unit displays the woman's predicted menstruation start date that was calculated by adding the number of days obtained based on the number of high temperature phase days to the woman's most recent ovulation date, in comparison to the woman's predicted menstruation start date that was calculated based on the woman's most recent menstruation start date and the number of menstrual cycle days. Accordingly, by looking at the display of the display screen, the user can be made aware of the fact that the predicted menstruation start date has been updated for increased precision.

A predicted menstruation start date calculation apparatus according to an embodiment further includes

a basal body temperature acquisition unit configured to acquire the woman's basal body temperature for each day,

wherein the date information acquisition unit acquires the woman's past menstruation start date or ovulation date by estimation based on the basal body temperature acquired by the basal body temperature acquisition unit, and uses the acquired past menstruation start date or ovulation date to calculate and acquire the number of menstrual cycle days and the number of high temperature phase days over a plurality of menstrual cycles of the woman.

With the predicted menstruation start date calculation apparatus according to this embodiment, the basal body temperature acquisition unit acquires the woman's basal body temperature for each day. Furthermore, the date information acquisition unit acquires the woman's past menstruation start date or ovulation date by estimation based on the basal body temperature acquired by the basal body temperature acquisition unit, and uses the acquired past menstruation start date or ovulation date to calculate and acquire the number of menstrual cycle days and the number of high temperature phase days over a plurality of menstrual cycles of the woman. Accordingly, for example, it is not time-consuming for the user to input date information such as the “menstruation start date” or the “number of menstrual cycle days” via the operation unit (touch panel, keyboard, mouse, etc.).

A program according to one or more embodiments of the claimed invention is

a program for causing a computer to execute:

a step of acquiring a number of menstrual cycle days and a number of high temperature phase days over a plurality of menstrual cycles of a woman;

a step of, based on whether or not the number of menstrual cycle days of the woman falls within a predetermined range and whether or not the number of high temperature phase days of the woman falls within a predetermined range, performing classification into a specific type indicating that the number of menstrual cycle days is unstable and the number of high temperature phase days is stable, and into one or more types other than the specific type; and

a step of, if the type into which the woman is classified by the type classification unit is the specific type, calculating a predicted menstruation start date by adding a number of days obtained based on the number of high temperature phase days to the woman's most recent ovulation date, and if the type into which the woman is classified by the type classification unit is a type other than the specific type, calculating the predicted menstruation start date based on the woman's most recent menstruation start date and the number of menstrual cycle days.

With the program according to one or more embodiments of the claimed invention, it is possible to cause a computer to execute processing for calculating a woman's predicted menstruation start date as follows.

That is to say, via the operation unit (touch panel, keyboard, mouse, etc.), for example, the computer first acquires the number of menstrual cycle days and the number of high temperature phase days estimated using transitions in the basal body temperature, over a plurality of menstrual cycles of the woman.

Next, based on whether or not the number of menstrual cycle days of the woman falls within a predetermined range and whether or not the number of high temperature phase days of the woman falls within a predetermined range, a central processing unit (CPU), for example, of the computer performs classification into a specific type indicating that the number of menstrual cycle days is unstable and the number of high temperature phase days is stable, and into one or more types other than the specific type. Here, it is assumed that the “predetermined range” for the number of menstrual cycle days and the “predetermined range” for the number of high temperature phase days are stored in a storage unit such as a memory, for example.

Next, if the type into which the woman is classified by the type classification unit is the specific type, the CPU, for example, of the computer calculates the predicted menstruation start date by adding a number of days obtained based on the number of high temperature phase days to the woman's most recent ovulation date. Here, if the type into which the woman is classified by the type classification unit is the specific type, the number of high temperature phase days of the woman is stable, and therefore the predicted menstruation start date can be calculated precisely. On the other hand, if the type into which the woman is classified by the type classification unit is a type other than the specific type, the CPU, for example, of the computer calculates the predicted menstruation start date based on the woman's most recent menstruation start date and the number of menstrual cycle days. Here, if the type into which the woman is classified by the type classification unit is, for example, a type indicating that the number of menstrual cycle days and the number of high temperature phase days are both stable, or a type indicating that the number of menstrual cycle days is stable and the number of high temperature phase days is unstable, the number of menstrual cycle days of the woman is stable, and therefore the predicted menstruation start date can be calculated precisely. Note that if the woman belongs to a type indicating that the number of menstrual cycle days and the number of high temperature phase days are both unstable, it is intrinsically difficult to precisely calculate the woman's predicted menstruation start date.

A program of an embodiment furthermore causes a computer to execute: a step of, if the woman belongs to the specific type, calculating the predicted menstruation start date based on the woman's most recent menstruation start date and the number of menstrual cycle days, and thereafter calculating the predicted menstruation start date by adding a number of days obtained based on the number of high temperature phase days to the woman's most recent ovulation date, and updating the predicted menstruation start date.

According to the program of this embodiment, if the woman belongs to the specific type, the computer calculates the predicted menstruation start date based on the woman's most recent menstruation start date and the number of menstrual cycle days, and thereafter calculates the predicted menstruation start date by adding a number of days obtained based on the number of high temperature phase days to the woman's most recent ovulation date, and updates the predicted menstruation start date. Accordingly, regardless of whether the woman belongs to the specific type or a type other than the specific type, the user can find out the next predicted menstruation start date on the most recent menstruation start date.

Furthermore, if the woman belongs to the specific type, the computer calculates the predicted menstruation start date by adding a number of days obtained based on the number of high temperature phase days to the woman's most recent ovulation date and updates the predicted menstruation start date. Accordingly, if the woman belongs to the specific type, the user can find out the next predicted menstruation start date on the day when the ovulation date is detected, with greater precision.

A body analysis apparatus according to one or more embodiments of the claimed invention is

a body analysis apparatus configured to analyze a body, wherein

the body autonomously transitions between a plurality of phases that each indicate an internal state of the body, and

a plurality of types relating to the transition between the phases exist,

the apparatus including:

a model storage unit configured to store a model corresponding to a type for each type relating to the transition between the phases;

an obtaining unit configured to obtain observation data by observing the transition between the phases of the body;

a detection unit configured to detect a type relating to the transition between the phases of the body based on the observation data; and

an analysis unit configured to read out the model corresponding to the type detected by the detection unit from the model storage unit and analyze the body using the read-out model.

In the present specification, the “types relating to the transition between the phases” refers to, in the case where an A phase and a B phase exist, types defined according to whether or not the number of days in which the body exhibits the A phase is stable (whether or not the number of days falls within a predetermined range) and whether or not the number of days in which the body exhibits the B phase is stable (whether or not the number of days falls within a predetermined range), for example.

The body analysis apparatus according to one or more embodiments of the claimed invention is premised on the fact that the body autonomously transitions between a plurality of phases that each indicate an internal state of the body, and the fact that a plurality of types relating to the transition between the phases exist. With the body analysis apparatus, the model storage unit stores a model corresponding to a type for each type relating to the transition between the phases. The obtaining unit obtains observation data by observing the transition between the phases of the body. The detection unit detects a type relating to the transition between the phases of the body based on the observation data. The analysis unit reads out the model corresponding to the type detected by the detection unit from the model storage unit and analyzes the body using the read-out model. Thus, with the body analysis apparatus, according to a type relating to the transition between the phases that each indicate an internal state of the body, the body is analyzed using the model corresponding to that type, and therefore the body can be analyzed precisely.

### Advantageous Effects of Invention

As is evident from the description above, with the predicted menstruation start date calculation device and program according to one or more embodiments of the claimed invention, it is possible to precisely calculate a predicted menstruation start date according to a type determined based on whether or not the number of menstrual cycle days of the woman who is the measurement subject is stable, and whether or not the number of high temperature phase days is stable.

Also, with the body analysis apparatus according to one or more embodiments of the claimed invention, a body can be analyzed precisely.

### BRIEF DESCRIPTION OF DRAWINGS

FIG. 1 is a block diagram showing an embodiment in which a predicted menstruation start date calculation apparatus of the invention is configured as a system on a network.

FIG. 2 is a block diagram showing a configuration of a smartphone included in the system.

FIG. 3 is a block diagram showing a configuration of a server included in the system.

FIG. 4 is a block diagram showing a configuration of a thermometer included in the system.

FIG. 5A is a diagram illustrating number of menstrual cycle days and number of high temperature phase days. FIG. 5B is a diagram showing typical basal body temperature transitions during a menstrual cycle.

FIG. 6 is a diagram showing an algorithm for calculating a predicted menstruation start date in the system.

FIG. 7 is a diagram showing four types into which a woman is classified based on whether or not the number of menstrual cycle days of the woman is stable, and whether or not the number of high temperature phase days is stable.

FIG. 8A is a diagram showing an example of a display displayed on a display screen of the smartphone while the basal body temperature of the woman is in a low temperature phase. FIG. 8B is a diagram showing an example of a display displayed on a display screen of the smartphone while the basal body temperature of the woman is in a high temperature phase.

FIG. 9A is a diagram showing correspondence between a predicted menstruation start date calculated using a second equation Eq. 2 and an actual menstruation start date in a case where the woman belongs to a specific type indicating that the number of menstrual cycle days is unstable and the number of high temperature phase days is stable. FIG. 9B is a diagram showing correspondence between a predicted menstruation start date calculated using a first equation Eq. 1 and an actual menstruation start date in a case where the woman belongs to a specific type indicating that the number of menstrual cycle days is unstable and the number of high temperature phase days is stable.

FIG. 10 is a diagram showing an overall operation flow for the server.

FIG. 11 is a diagram showing an operation flow for input processing of the smartphone.

### DETAILED DESCRIPTION OF INVENTION

Hereinafter, an embodiment of the invention will be described in detail with reference to the drawings.

FIG. 1 shows an example in which a predicted menstruation start date calculation apparatus serving as a body analysis apparatus of the invention is configured as a system of the embodiment (indicated overall by reference numeral 100) on a network. The system 100 includes a smartphone 200, a server 300, and a thermometer 400. The smartphone 200 and the thermometer 400 can communicate with each other using near field communication (NFC). The smartphone 200 and the server 300 can communicate with each other via a network 900.

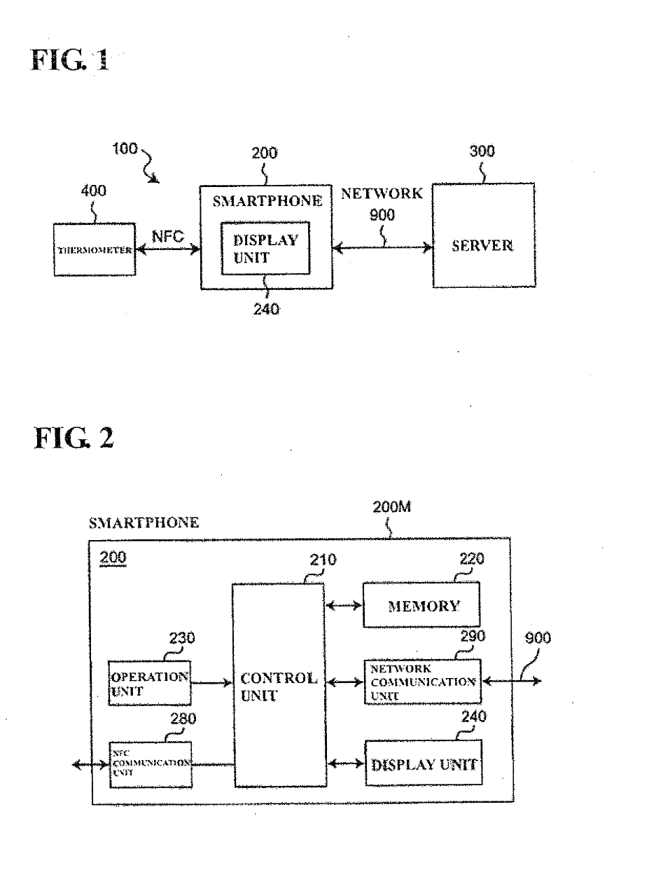

As shown in FIG. 2, the smartphone 200 includes a main body 200M, and a control unit 210, a memory 220, an operation unit 230, a display unit 240, an NFC communication unit 280, and a network communication unit 290, which are mounted in the main body 200M. The smartphone 200 is such that application software (a computer program) is installed on a commercially-available smartphone so as to cause it to perform later-described processing.

The control unit 210 includes a central processing unit (CPU)) and auxiliary circuits thereof, and controls the units of the smartphone 200 so as to execute later-described processing in accordance with programs and data stored in the memory 220. That is to say, data input from the operation unit 230 and the communication units 280 and 290 is processed, and the processed data is stored in the memory 220, displayed using the display unit 240, output from the communication units 280 and 290, or the like.

The memory 220 includes a random access memory (RAM) used as a work space that is needed for executing programs using the control unit 210, and a read only memory (ROM) for storing basic programs to be executed by the control unit 210. Also, a semiconductor memory (memory card, solid state drive (SSD)) or the like may be used as a storage medium of an auxiliary storage device for supplementing the storage region of the memory 220.

In this example, the operation unit 230 is comprised of a touch panel provided on the display unit 240. Note that a hardware operation device other than a keyboard may be included.

The display unit 240 includes a display screen (e.g., a liquid crystal display (LCD) or electroluminescence (EL) display, or the like). The display unit 240 is controlled by the control unit 210 so as to display a predetermined image on the display screen.

When the thermometer 400 is near the smartphone 200, the NFC communication unit 280 performs near field communication with the thermometer 400 and receives data indicating body temperature and measurement dates/times from the thermometer 400. Note that a Bluetooth low energy (BLE) communication unit may be included in the smartphone 200 and the thermometer 400 so that data can be transmitted in real time without needing to bring the smartphone 200 and the thermometer 400 close together.

The network communication unit 290 transmits information from the control unit 210 to another apparatus (in this example, the server 300) via the network 900, receives information transmitted from the other apparatus via the network 900, and transfers it to the control unit 210.

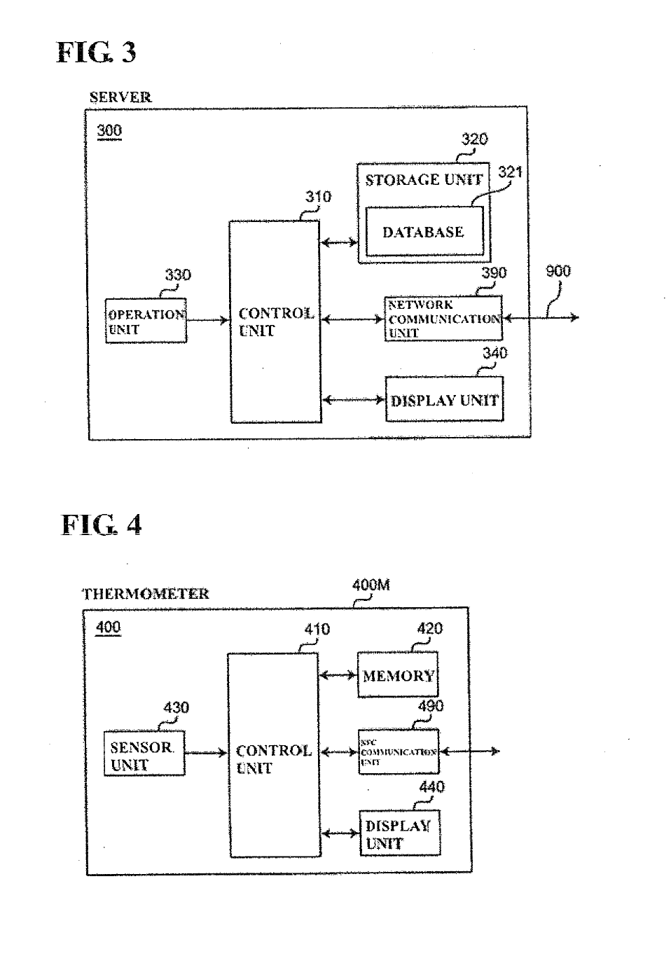

As shown in FIG. 3, the server 300 includes a control unit 310, a storage unit 320, an operation unit 330, a display unit 340, and a network communication unit 390. The server 300 has a program (software) installed in order to cause a general-use computer apparatus to perform later-described processing.

The control unit 310 includes a CPU (Central Processing Unit) and auxiliary circuits thereof, controls the units of the server 300 so as to execute predetermined processing in accordance with programs and data stored in the storage unit 320, processes data input from the operation unit 330 and the communication unit 390, and stores the processed data in the memory 320, displays it using the display unit 340, outputs it from the communication units 390, or the like.

The storage unit 320 includes a random access memory (RAM) used as a work space that is needed for executing programs using the control unit 310, and a read only memory (ROM) for storing basic programs to be executed by the control unit 310. The storage unit 320 is provided with a database 321 that includes body temperature measurement data sent from many users. As a model storage unit, the storage unit 320 stores later-described equations Eq. 1, Eq. 1′, and Eq. 2, which serve as models for calculating the predicted menstruation start date and predicted ovulation date. Also, a magnetic disk (hard disk (HD), flexible disk (FD)), an optical disk (compact disk (CD), digital versatile disk (DVD), Blu-ray disc (BD)), a magneto-optical disk (MO), a semiconductor memory (memory card, solid state drive (SSD)), or the like may be used as a storage medium of an auxiliary storage apparatus for supplementing the storage region of the storage unit 320.

In this example, the operation unit 330 is constituted by a keyboard and a mouse and inputs operation signals indicating operations performed by the user to the control unit 310. Also, the operation unit 330 may be constituted by another operation device such as a touch panel instead of or in addition to the keyboard and the mouse.

The display unit 340 includes a display screen (e.g., a liquid crystal display (LCD), electroluminescence (EL) display, or the like). The display unit 340 is controlled by the control unit 310 so as to display a predetermined image on the display.

The network communication unit 390 transmits information from the control unit 310 to another apparatus (in this example, the smartphone 200) via the network 900, receives information sent from the other apparatus via the network 900, and transfers it to the control unit 310.

As shown in FIG. 4, the thermometer 400 is a commercially-available electronic thermometer for feminine use in this example (MC-642L manufactured by OMRON) and includes a casing 400M, and a control unit 410, a memory 420, a sensor unit 430, a display unit 440, and an NFC communication unit 490, which are mounted in the casing 400M.

The sensor unit 430 includes a temperature sensor that measures and acquires body temperature, which serves as the internal state of the woman having the body. A woman's body temperature autonomously transitions between a high temperature phase and a low temperature phase in accordance with her menstrual cycle.

The memory 420 stores data of a program for controlling the thermometer 400, setting data for setting various functions of the thermometer 400, data of body temperature measurement results, and the like. In this example, the memory 420 can store a maximum of 40 days' worth of body temperature measurement data and measurement dates/times corresponding thereto. Also, the memory 420 is used as a work memory or the like for when a program is being executed.

The control unit 410 includes a CPU (Central Processing Unit) and controls the memory 420, the display unit 440 and the NFC communication unit 490 based on detection signals from the sensor unit 430, in accordance with a program for controlling the thermometer 400, which is stored in the memory 420. The display unit 440 includes a display screen constituted by an LCD (Liquid Crystal Display) element in this example, and displays predetermined information in accordance with a signal received from the control unit 410 on the display screen.

When the thermometer 400 is near the smartphone 200, the NFC communication unit 490 performs near field communication with the smartphone 200 so as to transmit data indicating the body temperature and measurement date/time to the smartphone 200. In this example, various types of setting information for the thermometer 400 (alarm (time/volume), buzzer sound setting, backlight intensity setting, and the like) are received from the smartphone 200.

The system 100 is used as follows.

i) A woman who is a user uses the thermometer 400 to measure basal body temperature each day over multiple menstrual cycles. In this example, a maximum of 40 days' worth of data of the measured basal body temperature is in the memory 420 of the thermometer 400 along with the measurement dates/times for when the basal body temperature was measured.

Each time the woman brings the thermometer 400 near the smartphone 200, as shown in step S201 of FIG. 11 (input processing), the NFC communication unit 280 and control unit 210 of the smartphone 200 function as a basal body temperature acquisition unit, and the smartphone 200 acquires data indicating the basal body temperature and measurement dates/times for each day over multiple menstrual cycles of the woman from the thermometer 400.

Note that if the woman uses a thermometer that does not include an NFC communication unit to measure her body temperature, the woman can manually input the data indicating the basal body temperature and measurement dates/times by operating the operation unit 230 of the smartphone 200.

ii) Also, as shown in step S202 of FIG. 11, the woman inputs the menstruation start dates for the multiple menstrual cycles as date information via the operation unit 230 of the smartphone 200. If the woman inputs the menstruation start dates by herself the actual menstruation start dates, which are not estimated, can be input, whereby the precision of calculating a later-described predicted menstruation start date can be increased.

iii) Also, as shown in step S203 of FIG. 11, the woman uses the operation unit 230 of the smartphone 200 to input other information. For example, “other information” indicates information for setting the current date/time, information for new user registration in the server 300, information indicating the model number of the thermometer 400.

Note that the inputting in steps S201 and S202 of FIG. 11 may be performed everyday by the woman who is the user, or, in this example, it may be performed each time the woman notices, within a maximum of 40 days. Also, steps S201 to S203 of FIG. 11 are not limited to the order in which they are described and any of them may be performed first.

v) After the input processing is performed in steps S201 to S203 of FIG. 11, the woman who is the user operates the operation portion 230 of the smartphone 200 so as to transmit the various types of input information from the network communication unit 290 to the server 300 via the network 900.

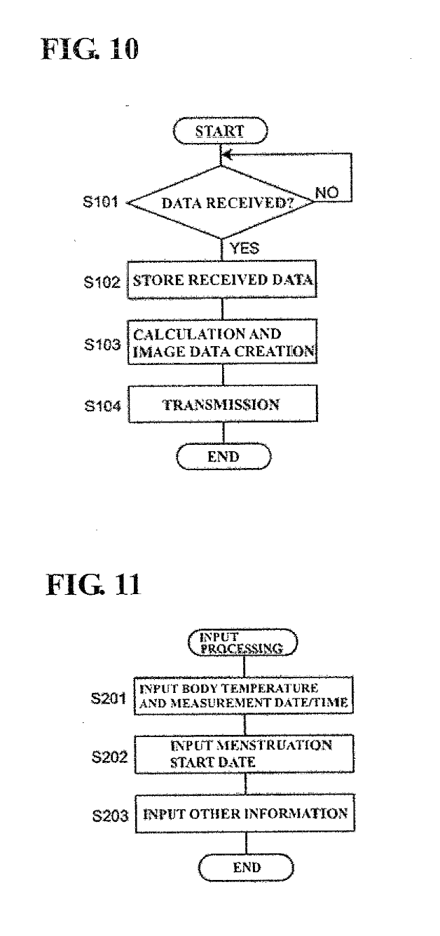

vi) As shown in step S101 of FIG. 10, the server 300 waits for the data from the smartphone 200 to be sent via the network 900. Upon receiving the data from the smartphone 200 from the network 900 via the network communication unit 390 (YES in step S101), the server 300 stores the received data in the storage unit 320 (step S102 in FIG. 10).

vii) Next, the server 300 performs calculation and image data creation (step S103 of FIG. 10). That is to say, the control unit 310 of the server 300 uses data from the smartphone 200, which is stored in the storage unit 320, to execute a series of processes including calculating the predicted menstruation start date as will be described later.

Also, each time a predicted menstruation start date is calculated, the server 300 creates data for an image to be displayed on the display unit 240 of the smartphone 200. Then, the created image data is stored in the database 321 and is transmitted from the network communication unit 390 to the smartphone 200 via the network 900 (step S104 of FIG. 10).

viii) The smartphone 200 receives the image data from the network 900 via the network communication unit 290 and stores it in the memory 220. Here, the woman who is the user operates the operation unit 230 of the smartphone 200 so as to select a menstruation start date prediction mode. Upon doing so, the control unit 210 of the smartphone 200 executes an operation of displaying the predicted menstruation start date on the display unit 240.

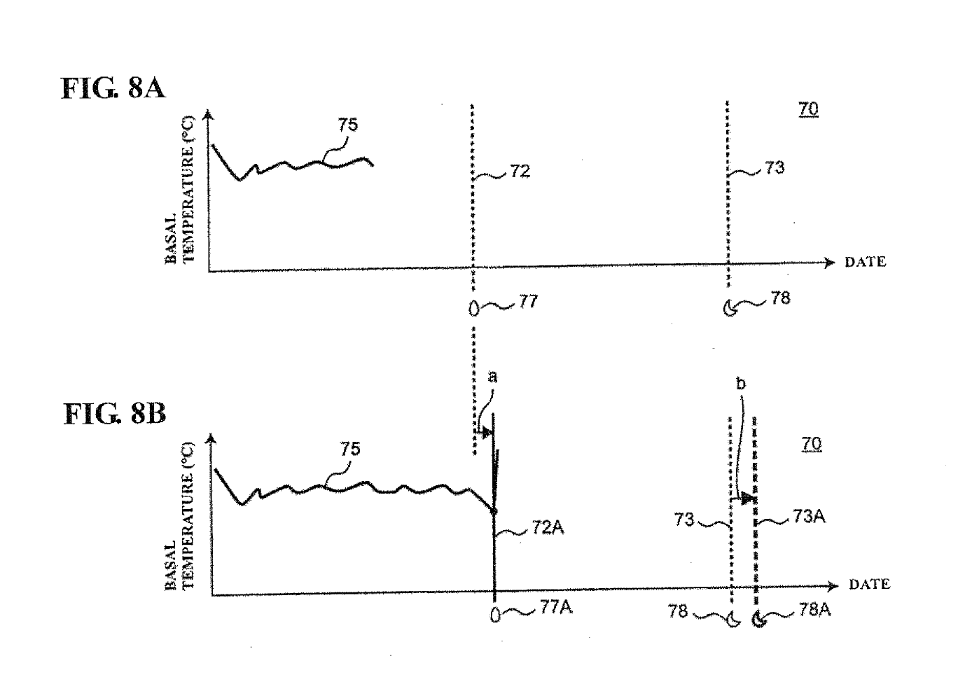

For example, as shown in FIG. 8A, on the display unit 240, the woman's basal body temperature for each day is displayed as a line graph 75 on a display screen 70 on which a vertical axis indicates the basal body temperature (° C.) and a horizontal axis indicates the date (calendar), and the predicted menstruation start date is displayed as a cursor line 73 constituted by a broken line that extends along the vertical direction. A crescent moon mark 78 serving as an icon that indicates a predicted menstruation start date is also displayed on the cursor line 73. Note that in this example, a cursor line 72 constituted by a broken line that indicates a predicted ovulation date calculated using the previous menstrual cycle is also displayed. An egg mark 77 serving as an icon that indicates the predicted ovulation date is also displayed on the cursor line 72. Accordingly, the user can easily find out the next predicted menstruation start date and predicted ovulation date by looking at the display of the display screen 70.

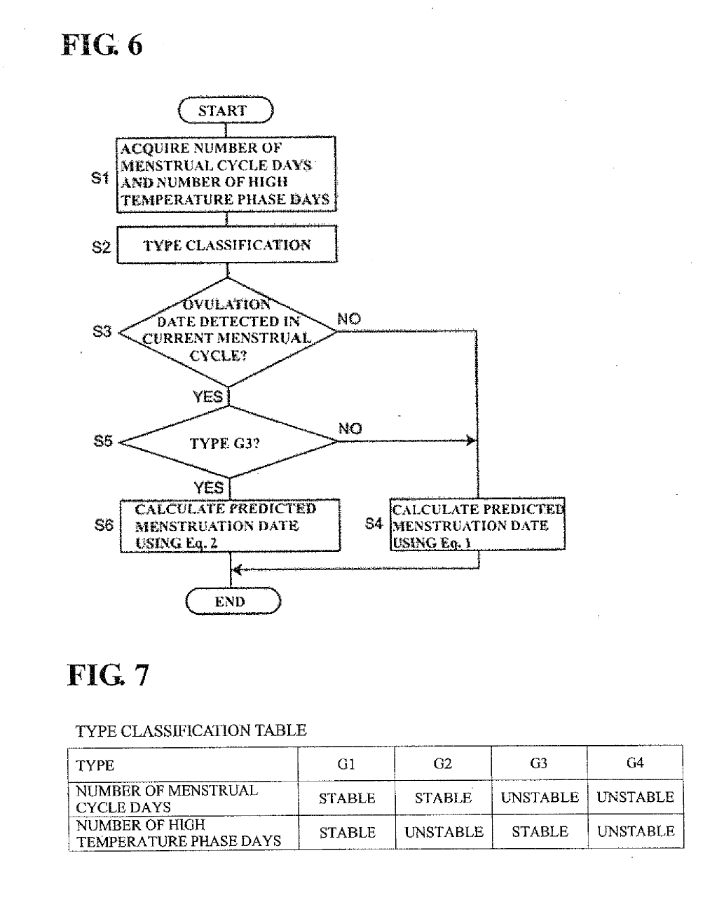

FIG. 6 shows a flow of a series of processes executed by the control unit 310 of the server 300.

As shown in step S1 of FIG. 6, the control unit 310 of the server 300 functions as a date information acquisition unit (or obtaining unit) that calculates and acquires the average number of menstrual cycle days and number of high temperature phase days over multiple menstrual cycles (in this example, the most recent three to five cycles) of the woman as observation data.

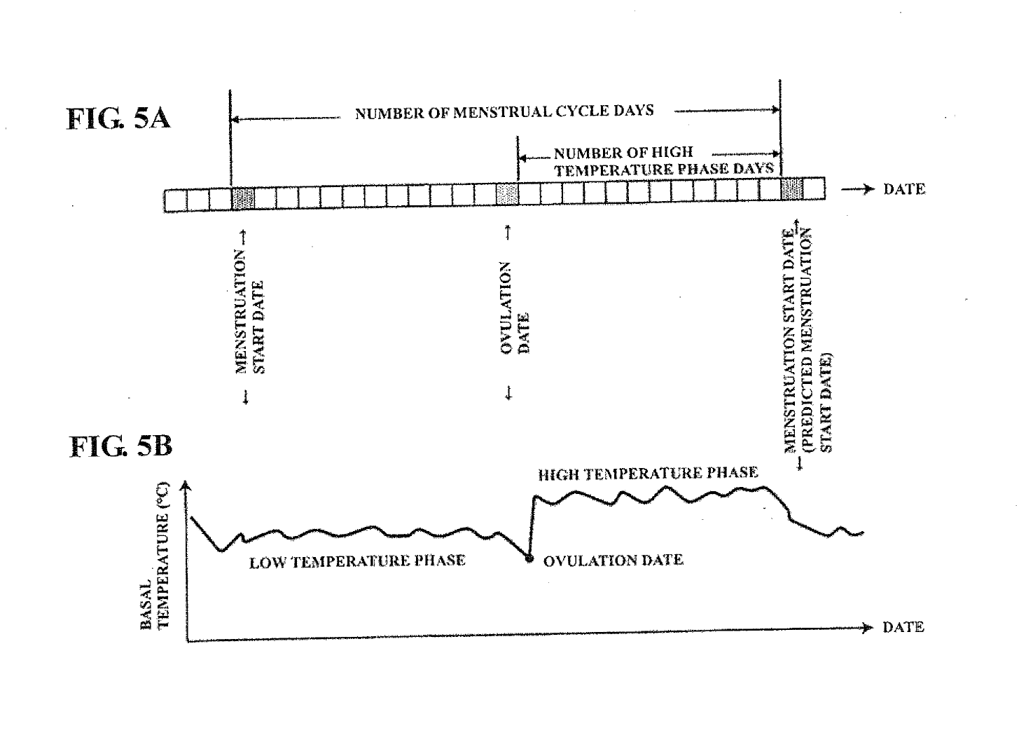

Here, as shown schematically in FIG. 5A, the number of menstrual cycle days is defined as the number of days from the menstruation start date (first day of menstrual cycle) to the day before the next menstruation start date (or predicted menstruation start date). The number of high temperature phase days is defined as a period during which the basal body temperature is at a high temperature level, as shown in FIG. 5B.

The day input in step S202 of FIG. 11 can be used as the woman's past menstruation start date that is to be the basis for calculating the number of menstrual cycle days. Note that the woman's past menstruation start date may be acquired by estimating using a known method, based on the basal body temperature input in step S201 of FIG. 11. For example, the menstruation start date can be estimated according to the date of transitioning from the high temperature phase to the low temperature phase. In such a case, the woman who is the user does not need to input the past menstruation start date via the operation unit 230 of the smartphone 200 and thus can be spared the burden of inputting it.

The woman's past ovulation date that is to be the basis for calculating the number of high temperature phase days is obtained by estimating using a known method, based on the basal body temperature input in step S201 of FIG. 11. For example, as can be understood from FIG. 5B, the ovulation date can be estimated as the day before the date of the transition from the low temperature phase to the high temperature phase.

Note that in this example, the server 300 (the control unit 310 thereof) calculates the number of menstrual cycle days and the number of high temperature phase days, but there is no limitation to this. The woman may input the average number of menstrual cycle days from the most recent three to five cycles by herself via the operation unit 230 of the smartphone 200, and then the server 300 may acquire the input data by receiving it from the smartphone 200. Also, if no data on the average number of high temperature phase days for the most recent three to five cycles exists, a standard “14 days” may be used as the average number of high temperature phase days.

Next, as shown in step S2 of FIG. 6, the control unit 310 of the server 300 functions as a type classification unit (or detection unit) that performs classification into (or detection of) types relating to the transition between phases, based on whether or not the dumber of menstrual cycle days of the woman falls within a predetermined range, and whether or not the number of high temperature phase days of the woman falls within a predetermined range.

Here, as the reference for whether or not the number of menstrual cycle days of the woman falls within a “predetermined range”, it is possible to use whether or not the standard deviation (S_{MM}) of the number of menstrual cycle days of the woman's most recent three to five cycles is less than or equal to a statistical survey value (2.90 days) determined by a gynecologist or the like, for example, or in other words, whether or not S_{MM}≦2.90 as the reference.

Also, as the reference for whether or not the number of high temperature phase days of the woman falls within a “predetermined range”, it is possible to use whether or not the standard deviation (S_{OM}) of the number of high temperature phase days of the woman's most recent three to five cycles is less than or equal to a statistical survey value (1.60 days) determined by a gynecologist or the like, for example, or in other words, whether or not S_{OM}≦1.60 as the reference.

Also, as shown in FIG. 7, examples of types into which the woman is classified include a first type G1 indicating that the number of menstrual cycle days and the number of high temperature phase days are both stable, a second type G2 indicating that the number of menstrual cycle days is stable and the number of high temperature phase days is not stable, a third type G3 indicating that the number of menstrual cycle days is not stable and the number of high temperature phase days is stable, and a fourth type G4 indicating that the number of menstrual cycle days and the number of high temperature phase days are both unstable. The third type G3 corresponds to a specific type, and the first type G1, the second type G2, and the fourth type G4 correspond to types other than the specific type.

Next, as shown in step S3 of FIG. 6, the control unit 310 of the server 300 determines whether or not the ovulation date was detected in the current menstrual cycle.

Specifically, basal body temperature data input in step S201 of FIG. 11 is searched for so as to detect whether or not the woman's basal body temperature has transitioned from the low temperature phase to the high temperature phase. If a transition from the low temperature phase to the high temperature phase has not yet been made (NO in step S3), the processing moves to step S4, which will be described later. On the other hand, if a transition from the low temperature phase to the high temperature phase has already been made (YES in step S3), the day before the day when the transition to the high temperature phase was made is detected as the ovulation date. Then, the processing moves to step S5, which will be described later.

The determination of whether or not the ovulation date was detected is performed when the data is received (YES in step S101 of FIG. 10).

In step S4 of FIG. 6, the control unit 310 of the server 300 functions as a calculation unit (or analysis unit) that reads out the next first equation Eq. 1 from the storage unit 320 and calculates the woman's predicted menstruation start date using the first equation Eq. 1, regardless of the type to which the woman belongs.

(Predicted menstruation start date)=(menstruation start date)+(number of menstrual cycle days) (Eq. 1)

Here, the “menstruation start date” on the right side of the equation is the most recent menstruation start date input in step S202 of FIG. 11. Also, the “number of menstrual cycle days” on the right side of the equation is the average number of menstrual cycle days from the most recent three to five cycles acquired in step S1 of FIG. 6.

Calculation by means of the first equation Eq. 1 is performed on the first day of the current menstrual cycle. This is because the transition from the low temperature phase to the high temperature phase has not yet been made (NO in step S3) on the first day of the menstrual cycle.

Accordingly, regardless of the type to which the woman belongs, the user can find out the next predicted menstruation start date on die first day of the current menstrual cycle (i.e., the most recent menstruation start date).

Here, if the woman belongs to the first type G1 (type indicating that the number of menstrual cycle days and the number of high temperature phase days are both stable) or the second type G2 (type indicating that the number of menstrual cycle days is stable and the number of high temperature phase days is unstable), for example, the number of menstrual cycle days of the woman is stable, and therefore the predicted menstruation start date can be calculated precisely using the first equation Eq. 1. On the other hand, if the woman belongs to the third type G3 (type indicating that the number of menstrual cycle days is unstable and the number of high temperature phase days is stable), the number of menstrual cycle days of the woman is unstable, and therefore the precision of the predicted menstruation start date is not favorable in this step. Also, if the woman belongs to the fourth type G4 (type indicating that the number of menstrual cycle days and the number of high temperature phase days are both unstable), it is intrinsically difficult to precisely calculate the predicted menstruation start date.

In this example, the control unit 310 of the server 300 also reads out a subsequent equation Eq. 1′ from the storage unit 320 and calculates the predicted ovulation date in the woman's next menstrual cycle using the equation Eq. 1′.

(Predicted ovulation date)=(predicted menstruation start date)−(number of high temperature phase days)−1 (Eq. 1′)

Here, the “predicted menstruation start date” on the right side of the equation is the date calculated using Eq. 1 in step S4 of FIG. 6. Also, the “number of high temperature phase days” on the right side of the equation is the average number of high temperature phase days of the most recent three to five cycles acquired in step S1 of FIG. 6.

As a result, display such as that shown in FIG. 8A is performed on the display unit 240 of the smartphone 200.

In step S5 of FIG. 6, the control unit 310 of the server 300 determines whether or not the woman belongs to the third type G3 (type indicating that the number of menstrual cycle days is unstable and the number of high temperature phase days is stable), which serves as the specific type among the four types G1 to G4.

Here, if the woman belongs to the third type G3 (YES in step S5), the processing moves to step S6, and the control unit 310 of the server 300 functions as a calculation unit (or analysis unit) that reads out the next second equation Eq. 2 corresponding to the third type G3 from the storage unit 320 and calculates and updates the woman's predicted menstruation start date using the second equation Eq. 2.

(Predicted menstruation start date)=(ovulation date)+(number of high temperature phase days)+1 (Eq. 2)

Here, the “ovulation date” on the right side of the equation is the most recent ovulation date acquired in step S1 of FIG. 6. Also, the “number of high temperature phase days” on the right side of the equation is the average number of high temperature phase days of the most recent three to five cycles acquired in step S1 of FIG. 6.

As a result, display such as that shown in FIG. 8B is performed by the display unit 240 of the smartphone 200. That is to say, on the display screen 70, a cursor line 73A constituted by a broken line indicating the predicted menstruation start date calculated using the second equation Eq. 2 in step S6 is displayed instead of the cursor line 73 constituted by a broken line indicating the predicted menstruation start date calculated using the first equation Eq. 1 in step S4. If the predicted menstruation start date calculated using the second equation Eq. 2 is different from the predicted menstruation start date calculated using the first equation Eq. 1, the cursor line 73A will be shifted as indicated by arrow b, for example, with respect to the cursor line 73.

Here, since the woman belongs to the third type G3, the number of high temperature phase days of the woman is stable. Accordingly, the woman's predicted menstruation start date can be calculated precisely using the second equation Eq. 2. As a result, the user can find out the next predicted menstruation start date with greater precision on the day when the ovulation date is detected.

In this example, the cursor line 73A is displayed boldly (and/or darker) for emphasis with respect to the cursor line 73. Accordingly, by looking at the display on the display screen 70, the user can be made aware of the fact that the predicted menstruation start date has been updated for greater precision.

Also, the cursor line 72A constituted by the solid line that indicates the ovulation date detected in step S3 (the ovulation date detected in the current menstrual cycle) is displayed on the display screen 70 instead of the cursor line 72 constituted by the broken line that indicates the predicted ovulation date. If the ovulation date detected in step S3 is different from the predicted ovulation date, the cursor line 72A will be shifted with respect to the cursor line 72, as indicated by arrow a, for example. Also, by looking at the cursor line 72A constituted by the solid line, the user can be made aware of the fact that the ovulation date was detected.

On the other hand, if the woman does not belong to type G3 and belongs to type G1, G2, or G4 in step S5 of FIG. 6 (NO in step S5), the processing once again moves to step S4, and the control unit 310 of the server 300 reads out the above-described first equation Eq. 1 that corresponds to type G1, G2, or G4 from the storage unit 320 and once again calculates the woman's predicted menstruation start date using the first equation Eq. 1.

In this case, display of the cursor line 73 constituted by the broken line that indicates the predicted menstruation start date calculated using the first equation Eq. 1 in step S4 is maintained on the display screen 70 of the smartphone 200.

Also, the cursor line 72A constituted by the solid line that indicates the ovulation date detected in step S3 (the ovulation date detected in the current menstrual cycle) is displayed on the display screen 70 instead of the cursor line 72 constituted by the broken line that indicates the predicted ovulation date. By looking at the cursor line 72A constituted by the solid line, the user can be made aware of the fact that the ovulation date was detected.

Thus, according to the predicted menstruation start date calculation apparatus, a predicted menstruation start date can be precisely calculated according to types G1 to G3, which are determined based on whether or not the number of menstrual cycle days of the woman who is the measurement subject is stable, and whether or not the number of high temperature phase days is stable. Note that as described above, if the woman belongs to the fourth group G4, it is intrinsically difficult to precisely calculate the predicted menstruation start date.

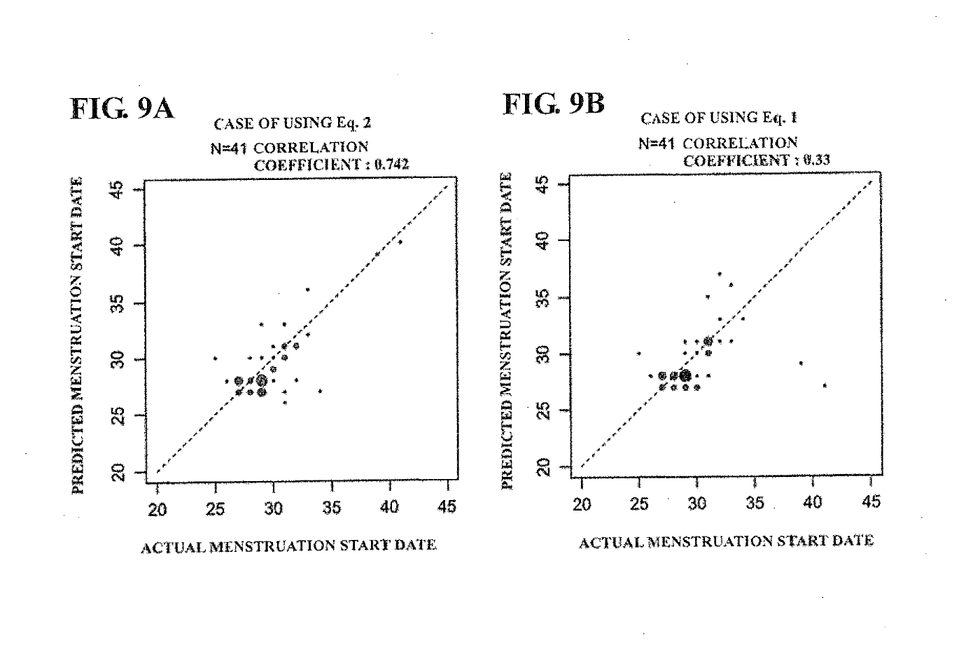

FIG. 9A shows a scatter plot for verifying the correspondence between the predicted menstruation start dates calculated using the second equation Eq. 2 and the actual menstruation start dates, using multiple women belonging to the third type G3 as measurement subjects. Also, FIG. 9B shows a scatter plot for verifying the correspondence between the predicted menstruation start dates calculated using the first equation Eq. 1 and the actual menstruation start dates, using multiple women belonging to the third type G3 as measurement subjects. In both FIG. 9A and FIG. 9B, the measurement subject number N=41. Also, in these scatter plots, one piece of data (a point of correspondence between one piece of predicted menstruation start date data and one piece of menstruation start date data) is represented by the circular mark with the smallest radius. If two, three, or more pieces of data overlap with each other, they are represented using double circles, triple circles, and the like respectively.

In FIG. 9A, the correlation coefficient of the predicted menstruation start dates calculated using the second equation Eq. 2 and the actual menstruation start dates is 0.742. In contrast to this, in FIG. 9B, the correlation coefficient between the predicted menstruation start date calculated using the first equation Eq. 1 and the actual menstruation start date is 0.33. Thus, if the woman belongs to the third type G3, it can be verified that the correspondence between the calculated predicted menstruation start date and the actual menstruation start date is improved. According to one or more embodiments of the claimed invention, it can be said that the predicted menstruation start date can be precisely calculated according to the types G1 to G3, which are determined based on whether or not the number of menstrual cycle days of the woman who is the measurement subject is stable, and whether or not the number of high temperature phase days is stable.

With the above-described embodiment, the woman's predicted menstruation start date and ovulation date (or predicted ovulation date) are displayed as icons (marks) or cursor lines on a calendar on the display screen 70, but there is no limitation to this. The woman's predicted menstruation start date may be displayed on the display screen 70 as characters indicating a date or a message, such as “Next predicted menstruation start date is April 1st”, for example. Also, when the predicted menstruation start date is updated using the second equation Eq. 2, display may be performed in which a mark (symbol) indicating that updating has been performed is attached to the characters indicating the predicted menstruation start date (date).

Also, in this embodiment, the predicted menstruation start date calculation apparatus of the present invention is configured as a system 100 on a network that includes a smartphone 200, a server 300, and a thermometer 400, but there is no limitation to this.

For example, the predicted menstruation start date calculation apparatus according to one or more embodiments of the claimed invention may be configured by only the thermometer 400 and the smartphone 200. In other words, it is assumed that the control unit 210, memory 220, operation unit 230, display unit 240, and network communication unit 290 of the smartphone 200 will fulfill the functions of the control unit 310, the storage unit 320, the operation unit 330, the display unit 340, and the network communication unit 390 of the server 300, in addition to their respective functions. In this case, a program for causing the control unit 210 to execute a method for calculating the woman's predicted menstruation start date is installed in the memory 220 of the smartphone 200. Accordingly, the predicted menstruation start date calculation apparatus according to one or more embodiments of the claimed invention can be configured to be smaller and more compact.

If the program causes the control unit 210 to execute the method for calculating the woman's predicted menstruation start date, the predicted menstruation start date can be precisely calculated according to types determined based on whether or not the number of menstrual cycle days of the woman is stable, and whether or not the number of high temperature phase days is stable.

The program can be recorded on a recording medium such as a CD, DVD, or a flash memory as application software. The application software recorded on the recording medium is installed on a substantial computer apparatus such as a smartphone, a personal computer, or a personal digital assistant (PDA), and thereby the computer apparatus can execute the method for calculating the woman's predicted menstruation start date.

Note that by furthermore incorporating the sensor unit 430 of the thermometer 400 in this kind of smartphone (in which the function of the server 300 is incorporated), the predicted menstruation start date calculation apparatus according to one or more embodiments of the claimed invention may be constituted by substantially only the smartphone.

Also, the predicted menstruation start date calculation apparatus according to one or more embodiments of the claimed invention may be configured using a laptop or a mobile phone having the same constituent elements and functions as these types of smartphones (smartphone in which the functions of the server 300 are incorporated, smartphone in which the sensor unit 430 of the thermometer 400 is further incorporated), instead of these types of smartphones.

In the example above, “types relating to phase transition” were four types defined according to whether or not the number of days during which the woman exhibits the high temperature phase is stable (whether or not it falls within a predetermined range), and whether or not the number of days during which the woman exhibits the low temperature phase is stable (whether or not it falls within a predetermined range). However, there is no limitation to this, and embodiments of the claimed invention can be widely applied to cases where a body autonomously transitions between multiple phases that each indicate the internal state of the body and multiple types relating to the above-described transition between the phases exist. Accordingly, the body can be analyzed with good precision.

### REFERENCE NUMERALS LIST

100 System
200 Smartphone
240 Display unit
300 Server
400 Thermometer

1. A predicted menstruation start date calculation apparatus, comprising:
a date information acquisition unit configured to acquire a number of menstrual cycle days and a number of high temperature phase days over a plurality of menstrual cycles of a woman;
a type classification unit configured to, based on whether or not the number of menstrual cycle days of the woman falls within a predetermined range and whether or not the number of high temperature phase days of the woman falls within a predetermined range, perform classification into a specific type indicating that the number of menstrual cycle days is unstable and the number of high temperature phase days is stable, and into one or more types other than the specific type; and
a calculation unit configured to, if the type into which the woman is classified by the type classification unit is the specific type, calculate a predicted menstruation start date by adding a number of days obtained based on the number of high temperature phase days to the woman's most recent ovulation date, and if the type into which the woman is classified by the type classification unit is a type other than the specific type, calculate the predicted menstruation start date based on the woman's most recent menstruation start date and the number of menstrual cycle days.

2. The predicted menstruation start date calculation apparatus according to claim 1, wherein
if the woman belongs to the specific type, the calculation unit calculates the predicted menstruation start date based on the woman's most recent menstruation start date and the number of menstrual cycle days, and thereafter calculates the predicted menstruation start date by adding a number of days obtained based on the number of high temperature phase days to the woman's most recent ovulation date, and updates the predicted menstruation start date.

3. The predicted menstruation start date calculation apparatus according to claim 1, further comprising:
a display unit configured to display the woman's predicted menstruation start date on a display screen as characters indicating a date or as an icon on a calendar.

4. The predicted menstruation start date calculation apparatus according to claim 3, wherein
in an emphasized manner on the display screen, the display unit displays the woman's predicted menstruation start date that was calculated by adding the number of days obtained based on the number of high temperature phase days to the woman's most recent ovulation date, in comparison to the woman's predicted menstruation start date that was calculated based on the woman's most recent menstruation start date and the number of menstrual cycle days.

5. The predicted menstruation start date calculation apparatus according to claim 1, further comprising:
a basal body temperature acquisition unit configured to acquire the woman's basal body temperature for each day,
wherein the date information acquisition unit acquires the woman's past menstruation start date or ovulation date by estimation based on the basal body temperature acquired by the basal body temperature acquisition unit, and uses the acquired past menstruation start date or ovulation date to calculate and acquire the number of menstrual cycle days and the number of high temperature phase days over a plurality of menstrual cycles of the woman.

6. A non-transitory computer readable medium having stored thereon a program for causing a computer to execute:
acquiring a number of menstrual cycle days and a number of high temperature phase days over a plurality of menstrual cycles of a woman;
based on whether or not the number of menstrual cycle days of the woman falls within a predetermined range and whether or not the number of high temperature phase days of the woman falls within a predetermined range, performing classification into a specific type indicating that the number of menstrual cycle days is unstable and the number of high temperature phase days is stable, and into one or more types other than the specific type; and
if the type into which the woman is classified by the type classification unit is the specific type, calculating a predicted menstruation start date by adding a number of days obtained based on the number of high temperature phase days to the woman's most recent ovulation date, and if the type into which the woman is classified by the type classification unit is a type other than the specific type, calculating the predicted menstruation start date based on the woman's most recent menstruation start date and the number of menstrual cycle days.

7. The non-transitory computer readable medium having stored thereon the program according to claim 6 for causing a computer to further execute:
a step of, if the woman belongs to the specific type, calculating the predicted menstruation start date based on the woman's most recent menstruation start date and the number of menstrual cycle days, and thereafter calculating the predicted menstruation start date by adding a number of days obtained based on the number of high temperature phase days to the woman's most recent ovulation date, and updating the predicted menstruation start date.

8. A body analysis apparatus configured to analyze a body, wherein
the body autonomously transitions between a plurality of phases that each indicate an internal state of the body, and
a plurality of types relating to the transition between the phases exist,
the apparatus comprising:
a model storage unit configured to store a model corresponding to a type for each type relating to the transition between the phases;
an obtaining unit configured to obtain observation data by observing the transition between the phases of the body;
a detection unit configured to detect a type relating to the transition between the phases of the body based on the observation data; and
an analysis unit configured to read out the model corresponding to the type detected by the detection unit from the model storage unit and analyze the body using the read-out model.
